(19) United States
(12) Patent Application Publication
(10) Pub. No.: US 2025/0114025 A1
(43) Pub. Date: Apr. 10, 2025

(54) METHOD FOR ANALYZING ARRYTHMIA

(71) Applicant: CORIFY CARE, S.L., Madrid (ES)

(72) Inventors: Ruben MOLERO ALABAU, Valencia (ES); Carlos FAMBUENA SANTOS, Valencia (ES); Javier MILAGRO SERRANO, Valencia (ES); Andreu MARTINEZ CLIMENT, Valencia (ES); Maria GUILLEM SANCHEZ, Valencia (ES)

(21) Appl. No.: 18/688,681

(22) PCT Filed: Sep. 2, 2022
(86) PCT No.: PCT/EP2022/074485

§ 371 Date: Mar. 1, 2024

(30) Foreign Application Priority Data
Sep. 3, 2021 (EP) 21382797.5

(51) Int. Cl. A61B 5/364; A61B 5/367; G16H 10/60; G16H 50/30

(52) U.S. Cl. CPC A61B 5/364; A61B 5/367; G16H 10/60; G16H 50/30

(57) ABSTRACT

The present invention belongs to the field of computer implemented methods for the quantification of arrhythmia complexity.

The present invention is particularly related to a method for obtaining the reproducibility score (RS) of any type of cardiac arrhythmia.

### TECHNICAL FIELD OF THE INVENTION

The present invention belongs to the field of computer implemented methods for the quantification of arrhythmia complexity.

The present invention is particularly related to a method for obtaining the reproducibility score (RS) of any type of cardiac arrhythmia.

### BACKGROUND OF THE INVENTION

Currently, abnormalities of cardiac rhythm, i.e., cardiac arrhythmias, are prevalent in adults, affecting >2% of individuals, and present an incidence of 0.5% per year, which is similar to rates of other severe cardiac conditions, such as stroke, myocardial infarction or heart failure.

Additionally, the risk of arrhythmia is increased in the setting of older age, male sex, traditional cardiac risk factors, chronic kidney disease, and heart failure. Thus, the correct detection of cardiac arrhythmias and a proper clinical management is of paramount importance, and the tendency towards personalized medicine encourages the development of methodologies for the individualized therapeutic strategy for each patient.

The most common approach in the state of the art for the assessment of cardiac arrhythmias is the analysis of surface electrocardiographic (ECG) signals. An ECG signal is a bioelectric signal describing the electrical activity of the heart, measured at the torso surface. ECG signals result from the spatiotemporal combination of the electrical activity of each cell of the cardiac tissue, and present characteristic waves which are associated with the different stages of the cardiac cycle. For example, the P wave, the QRS complex or the T wave, are related with atrial depolarization, ventricular depolarization and ventricular repolarization, respectively. Therefore, ECG signals contain large information on cardiac electrical activity, and their analysis remains the principal approach for the diagnosis of cardiac dysfunction, including cardiac arrhythmias.

However, the analysis of the surface ECG signals has drawbacks, since only a few surface signals are registered at the torso of the patient. Therefore, the ECG does not provide information of the actual cardiac electrical activity in the heart, neither of the rotors which maintain the abnormal electrical activity of the arrhythmia, which is of paramount importance in the understanding of the pathophysiological mechanisms sustaining the arrhythmia.

As a second option, and in order to have information of cardiac electrical activity within the heart, the state-of-the-art approach at the electrophysiology units of the hospitals is the use of invasive mapping systems. Essentially, these systems register intracavitary electrical signals using one or several invasive catheters, which are placed within the cardiac chamber to be analyzed. Afterwards, the acquired signals are processed to provide relevant information on the status of cardiac electrical system.

However, such invasive mapping systems have several drawbacks. First of all, since the use of a catheter is required, only a narrow region of the cardiac tissue can be assessed simultaneously. Additionally, such systems are expensive and have several associated risks, implying procedures which may last several hours. For these reasons, only a few patients have access to these mapping systems and analysis procedures.

A third possibility for the analysis of cardiac activity is electrocardiographic imaging (ECGI). ECGI consists on the noninvasive estimation of the electrical activity at the surface of the heart using only surface ECG signals, and geometric models of the patient's torso and heart. In difference with invasive mapping systems, ECGI is noninvasive in nature, and allows to visualize the electrical activity of the whole cardiac tissue simultaneously. Moreover, its associated risks are the same than that of a conventional ECG (i.e., lower than invasive mapping systems), being cheaper than invasive mapping systems both economic- and time-wise.

Notwithstanding the aforementioned analysis systems, cardiac electrical activity during arrhythmia is also evaluated based on certain metrics which allow to summarize and understand it. Some examples of these state-of-the-art metrics are the conduction velocity of the cardiac tissue or the quantification of reentrant circuits. The aim of these metrics is twofold: to locate the tissue region sustaining the arrhythmia and to stratify the severity of the arrhythmia. In both cases, the studied metrics are obtained from single time intervals, thus not having reproducibility into account. However, some arrhythmias, e.g., atrial fibrillation, are irregular in nature, thus implying rapid variations on the measured electrical signals and therefore in the metrics extracted from them. In this way, the interpretation derived from the analysis of such metrics is greatly dependent on the time interval from which they are calculated. Moreover, there are discrepancies in the scientific community regarding the use of said metrics as a measure of arrhythmia complexity.

Additionally, state-of-the-art approaches for the evaluation of cardiac electrical activity are mainly based on instantaneous metrics which only take into account a short time interval, and consequently, metrics are dependent on the selected analysis interval.

Therefore, reproducibility is not considered and arrhythmia complexity cannot be properly quantified.

Known documents have however described methodologies based on the analysis of different time intervals, aiming at using the variation of certain features among such intervals to stratify arrhythmias. An example is the approach disclosed in U.S. Pat. No. 9,060,699B2, in which the residuals from beat templates at two different time intervals are employed.

Nevertheless, derived information is only based in ECG morphological features derived from traditional 12-lead ECG, thus not providing information of electrical activity in the surface of the heart.

Another example is disclosed in US2020375490A1, in which correlation of electrical activity acquired at two different time intervals using an implantable device is analyzed for arrhythmia classification. However, the use of implantable technology is invasive in nature and have several associated risks, and the scope of said document is limited to the classification of arrhythmia, and does not aim at stratifying the complexity of a given arrhythmia type.

A third example is disclosed in WO2008035070A2, in which the analysis of dominant frequencies is addressed. In this document, a temporal stability index is presented as the average variation of the dominant frequency on successive time periods, intended to determine which portions of the cardiac tissue present a higher spatiotemporal variability.

In conclusion, existent known methodologies for arrhythmia characterization are nowadays not considering temporal variability of the analyzed features as they are based on single time measurements, or are not precise enough to study variations in cardiac electrical activity complexity, therefore providing an incomplete description of the arrhythmic behavior.

### SUMMARY OF THE INVENTION

The present invention provides a solution for the aforementioned problems, by means of a method according to claim 1. In dependent claims, preferred embodiments of the invention are defined.

The present invention allows to establish a level of confidence of the analyzed metrics for obtaining the reproducibility score of cardiac arrhythmia to quantify the cardiac arrhythmia complexity level of any kind. In this way, variability in the metrics can be threshold to establish a level of confidence of each metric.

Therefore, the present invention is intended to overcome the drawbacks of the current arrhythmia complexity determination techniques by providing a methodology for the reproducibility analysis of the metrics of cardiac electrical activity during arrhythmia.

Quantifying the reproducibility of such metrics is key to establish a level of confidence of the analyzed metrics, given that nonreproducible metrics can lead to a wrong interpretation depending on the selected analysis interval. Additionally, said quantification also provides a reproducibility score indicative of the arrhythmia complexity level.

Thus, the present invention provides a reproducible solution for the analysis of the metrics of cardiac electrical activity during arrhythmia, which improves the reliability of the cardiac arrhythmia assessment methods and techniques known in the art. The invention is based on the quantification of the reproducibility of relevant metrics of cardiac electrical activity.

In a first inventive aspect, the invention provides a computer implemented method for obtaining the reproducibility score (RS) of cardiac arrhythmia, the method comprising the following steps:
a) collecting as input data a geometrical model of the cardiac surface of a patient, the cardiac surface comprising a plurality N ∈ [symbol] and the cardiac electrical activity at the plurality N of nodes of said cardiac surface of a patient,
b) computing from the input data of step a), during a first time interval t₁, a number M of metrics designated as θ_{m,t}_{₁}, wherein m=1, . . . M,
c) computing from the input data of step a), during a second time interval t₂ which does not overlap with the first time interval t₁, the M metrics designated as θ_{m,t}_{₂} wherein m=1, . . . M,
d) determining the temporal variability Δθₘ of each of the M metrics computed, wherein m=1, . . . M, and
e) obtaining a reproducibility score (RS) as a combination of the temporal variability Δθₘ of each of the m metrics computed, wherein m=1, . . . M, being each temporal variability Δθₘ weighted by a corresponding weighting coefficient.

Throughout all this document, reproducibility is understood as the ability for the disclosed methodology of replicating the obtained result with a high degree of agreement when the proceeding is repeated, e.g., in different time intervals, thus meaning that the result is not depending on the time interval in which the electrical activity data is taken.

Thus, a reproducibility score is obtained as a result, which is indicative of the level of confidence of the analyzed metrics as well as the complexity level of the studied cardiac arrhythmia, understood as the regularity of the electrical activity during the arrhythmia (more complex meaning less regular arrhythmia).

Moreover, the reproducibility score provides valuable information on the complexity of the arrhythmia, being a higher RS representative of a less complex arrhythmia.

This value, when combined with machine learning techniques, can be used to guide the clinical practice in an individualized manner, thus aiding in the selection of the best treatment for each patient.

The method of the invention considers information of cardiac geometry of the patient. The knowledge of cardiac geometry for the computation of cardiac activity metrics is useful for the characterization of cardiac arrhythmias. On one hand, knowing the cardiac geometry allows the use of geometrical transformations or mathematical tools to compute certain metrics of interest in a more efficient way. On the other hand, the geometrical characteristics of the cardiac geometry are used for the computation of certain metrics (e.g., conduction velocity). This allows a better characterization of cardiac arrhythmia.

Advantageously, the present method is able to obtain the reproducibility score of any kind of arrhythmia, either regular or irregular. Moreover, the present method is also able to be applied to other fields of the technique, given that a person skilled in the art of data analysis understands that the present methodology of this inventive concept allows to compute a reproducibility score (RS) for whichever set of features that can be calculated in two different time intervals, by adjusting the required input data as well as the metrics to be measured. This is of application in a variety of scenarios, even unrelated to the field of cardiac arrhythmia.

Some nonlimiting applications of the described methodology are the analysis of heat maps, weather forecast, biometric systems or the analysis of time series.

According to the present method, the first and second time intervals, t₁ and t₂, are non-overlapping time intervals, which do not have the same origin. Such first and second time intervals, t₁ and t₂, can be either consecutive or non-consecutive.

In a particular embodiment, the first and second time intervals, t₁ and t₂, are non-consecutive time intervals.

In a particular embodiment, the first and second time intervals, t₁ and t₂, are time intervals of the same duration.

In a particular embodiment, the first and second time intervals, t₁ and t₂, can be separated by one to ten minutes. In a particular embodiment the first and second time intervals, t₁ and t₂, can be separated by five minutes.

In a particular embodiment, the combination of the temporal variability Δθₘ by means of weighting coefficients allows linear or n-polynomic combinations, neural networks, or any distribution of the weighting coefficients which allows the obtention of a reproducibility score (RS).

In a particular embodiment, in step e) the reproducibility score (RS) is obtained by a n^{th} order polynomic combination of the temporal variability Δθₘ of each of the M metric computed, weighted by respective weighting coefficients.

In a particular embodiment, in step e) the reproducibility score (RS) is obtained by combining the temporal variability Δθₘ of each of the M metric computed using neural networks.

In a particular embodiment, the reproducibility score (RS) of step e) is obtained according to the following expression:

$$R S = {α}_{1} Δ {θ}_{1} + {α}_{2} Δ {θ}_{2} + … + {α}_{M} Δ {θ}_{M} ,$$

wherein aₘ is the weighting coefficient for the m^{th} metric and Δθₘ is the temporal variability of the m^{th} metric computed.

Advantageously, such a combination of the temporal variability Δθₘ of each of the m metrics computed, wherein m=1, . . . M provides an easier and more efficient resolution of the value of the reproducibility score (RS).

In a particular embodiment, the weighting coefficients for the m^{th} metric aₘ can all be equivalent, so that each of the metrics considered contributes equally to the calculation of reproducibility score (RS). In another embodiment, the weighting coefficients aₘ for the m^{th} metric can have different values and be computed based on patient characteristics, such as physical characteristics (e.g., sex, body mass index, heart size) or patient condition (e.g., type of arrhythmia).

In a particular embodiment, the weighting coefficients for each of the M computed metrics are calculated based on patient characteristics, such as physical characteristics or patient condition.

In a particular embodiment, the temporal variability Δθₘ of the m^{th} metric is obtained at least by one of the following expressions:

$$Δ {θ}_{m} = \vert {θ}_{m , {t}_{1}} - {θ}_{m , {t}_{2}} \vert , or$$

$${Δθ}_{m} = \frac{\vert {θ}_{m , {t}_{1}} - {θ}_{m , {t}_{2}} \vert}{\vert {θ}_{m , {t}_{1}} \vert} , or$$

$${Δθ}_{m} = \frac{\vert {θ}_{m , {t}_{1}} - {θ}_{m , {t}_{2}} \vert}{\vert {θ}_{m , {t}_{2}} \vert} , or$$

$${Δθ}_{m} = {\vert {θ}_{m , {t}_{1}} - {θ}_{m , {t}_{2}} \vert}^{p} , p ∈ , or$$

$${Δθ}_{m} = 2 \cdot \frac{\vert {θ}_{m , {t}_{1}} - {θ}_{m , {t}_{2}} \vert}{\vert {θ}_{m , {t}_{1}} + {θ}_{m , {t}_{2}} \vert} ,$$

wherein θ_{m,t}_{₁} and θ_{m,t}_{₂} are the values of the m^{th} metric obtained from the first and second time intervals, t₁ and t₂, respectively.

Moreover, the temporal variability Δθₘ can be calculated by means of any equation that describes the difference or variation of a metric between two time intervals, particularly between the first and second time intervals, t₁ and t₂.

In step a) of the present method, part of the required input data are a geometrical model of the cardiac surface of a patient. Such a cardiac surface comprises a plurality of nodes, particularly N E N nodes, which are points of said cardiac surface wherein analysis is to be performed, particularly points by means of which the cardiac surface can be defined, and which contain specific information of the cardiac surface. Said nodes can be identified by means of their spatial position within the surface, and adjacent nodes of each of the different N nodes can be identified and numbered.

In a particular embodiment, the geometric model of the cardiac surface of step a) is obtained by segmentation of medical images or by adjustment of a mathematical model.

Particularly, the required cardiac surface and electrical activity can be directly obtained via ECGI systems, i.e., from the combination of geometrical models of a patient's torso and heart and surface ECG signals from a torso surface.

In a particular embodiment, the geometrical model of the patient's torso and the geometrical model of the heart can be obtained by segmentation of medical images acquired using medical and/or from a database of models. Additionally, the segmentation of medical images can be automatic, semi-automatic and/or manual. Moreover, the images can be acquired by segmentation of medical images acquired using medical imaging systems, such as MRI or CT scan.

In a particular embodiment, the torso geometry of the patient is obtained by a nonmedical imaging system. Thus, the non-medical imaging system generates a 3D geometric model of the patient's torso from a video recording or a set of images, therefore achieving the geometrical model of the patient's torso, and thus the cardiac surface, and the geometrical model of the heart.

The geometrical models of the patient's torso and heart can also be obtained from a database of models, representing different subject characteristics, such as physical characteristics (e.g., height or weight), disease states or gender.

Step a) of the present method also requires the electrical activity at the different N nodes of such cardiac surface, thus being the nodes the points wherein the electrical activity is measured.

In a particular embodiment, the electrical activity at several nodes, present on the surface of the patient's torso, can be registered by ECG signals, which can be acquired from a predetermined number of locations on the whole patient's torso surface using e.g., biosignals acquisition systems.

In a particular embodiment, the biosignal acquisition system comprises a combination of a high-density sensor vest and a biopotentials amplifier. More particularly, the high-density sensor vest can comprise from 100 to 150 electrodes homogeneously distributed along its surface, to be placed on the patient's torso.

In a particular embodiment, when the patient has e.g., a reduced size, a lower number of electrodes can be employed.

In a particular embodiment, the position of each sensor or electrode can be localized manually, or using semi-automatic or automatic detection systems or algorithms. Particularly, the position of each electrode is localized using automatic image recognition-based detection systems. The acquired signals are then conducted through a biopotentials amplifier where they can be amplified, digitized and sent to a workstation, which particularly can be a computer or a computing device or processing unit specifically designed for this purpose; or a computer readable medium, such as flash memories, optical media, such as CD-ROM, magnetic media, such as hard disks or floppy disks, or any other storage media which is intended to be read by a computer. In a particular embodiment of the invention, the workstation is a computer.

In a particular embodiment, the cardiac electrical activity at the plurality N of nodes of step a) is collected with at least one catheter placed within the cardiac chambers.

Advantageously, this enhances precision of regions of the cardiac tissue causing or being responsible of the maintenance of the arrhythmia, so that electrical activity at several points of the cardiac tissue can be analyzed in detail with the invasive catheter.

In a particular embodiment, the cardiac electrical activity at the plurality N of nodes of step a) is obtained by solving the inverse problem by means of the computing on a torso geometry model of a patient.

Particularly, such inverse problem is the inverse problem of electrocardiography.

The resolution of such inverse problem, in any way, provides an estimate epicardial electric activity on the cardiac surface (epicardium).

The inverse problem of electrocardiography is a mathematical problem which relates the electrical activity at each point of the cardiac surface (U_{H}) with the ECG signals at each point of the torso surface (U_{T}), by means of a transfer matrix (M). The inverse problem is thus formulated as:

MU_{H}=U_{T}

In a particular embodiment, the inverse problem can be mathematically solved as shown.

The transfer matrix can be calculated from the torso and the cardiac geometric models by using e.g., propagation models, such as the boundary elements method. Once the transfer matrix has been obtained, U_{H} can be estimated from U_{T} by obtaining the inverse transfer matrix.

In a particular embodiment, the epicardial potentials is calculated by minimizing the following equation:

$${‖ {MU}_{H} - {U}_{T} ‖}^{2} + λ {‖ {BU}_{H} ‖}^{2} ,$$

where λ is a regularization parameter and B is a spatial regularization matrix.

In a particular embodiment, the epicardial potentials can be obtained by applying methodologies for the resolution of the inverse problem such as the MFS method or methods based on singular value decomposition or Bayesian estimations.

In a particular embodiment, both U_{T} and U_{H} can undergo additional signal processing stages for, e.g., eliminating non-physiological signal components, such as the powerline interference, or the cancellation of certain cardiac signals. Also, U_{T} and U_{H} can undergo additional signal processing stages for the cancellation of certain cardiac signals.

In a particular embodiment, the cardiac surface comprising the plurality n of nodes in step a) is mapped to a 2D surface comprising the plurality of nodes by means of conformal mapping.

In a particular embodiment, such mapping is performed by stereographic projection, or by a Mercator projection.

In a particular embodiment, at least one metric is selected from: activation times, conduction velocity, voltage, phase, number of phase singularities, number of rotors, mean rotor duration, spatial entropy, slew rate, dominant frequency, repolarization times, or any other metric which can be of interest for the characterization of cardiac activity.

In a particular embodiment, the number of metrics M employed for computing the reproducibility score (RS) is greater than 1 (i.e.,M>1).

In a particular embodiment, the present method is applied with the particular number of metrics of M=2.

In a particular embodiment, M=2 and:
θ_{1,t}_{₁}=RD₁, is the mean rotor duration at the first time interval t₁,
θ_{2,t}_{₁}=SE₁, is the spatial entropy of the rotor histogram at the first time interval t₁,
θ_{1,t}_{₂}=RD₂, is the mean rotor duration at the second time interval t₂,
θ_{2,t}_{₂}=SE₂, is the spatial entropy of the rotor histogram at the second time interval t₂.

In a particular embodiment, the reproducibility score (RS) fulfills the following expression:

$$R S = {α}_{1} Δ SP + {α}_{2} Δ RD ,$$

wherein a₁, and a₂ are weighting coefficients and wherein ΔRD and ΔSE are the temporal variabilities of the mean rotor duration and the spatial entropy of the rotor histogram correspondingly.

In a particular embodiment, the present method is applied with the particular number of metrics of M=3.

In a particular embodiment, the present method is applied with the particular number of metrics of M=3, and wherein:
θ_{1,t}_{₁}=NPS₁, is the number of phase singularities at the first time interval t₁,
θ_{2,t}_{₁}=RD₁, is the mean rotor duration at the first time interval t₁,
θ_{3,t}_{₁}=SE₁, is the spatial entropy of the rotor histogram at the first time interval t₁,
θ_{1,t}_{₂}=NPS₂, is the number of phase singularities at the second time interval t₂,
θ_{2,t}_{₂}=RD₂, is the mean rotor duration at the second time interval t₂,
θ_{3,t}_{₂}=SE₂, is the spatial entropy of the rotor histogram at the second time interval t₂.

In a particular embodiment, the reproducibility score (RS) fulfills the following expression:

$$R S = {α}_{1} Δ N P S + {α}_{2} Δ R D + {α}_{3} Δ S E ,$$

wherein a₁, a₂ and a₃ are weighting coefficients and wherein ΔNPS, ΔRD and ΔSE are the temporal variabilities of the number of phase singularities, the mean rotor duration and the spatial entropy of the rotor histogram correspondingly.

In a particular embodiment, the present method is applied with the particular number of metrics of M=3, and wherein:
θ_{1,t}_{₁}=NPS₁, is the number of phase singularities at the first time interval tᵢ,
θ_{2,t}_{₁}=RD₁, is the mean rotor duration at the first time interval t₁,
θ_{3,t}_{₁}=SE₁, is the spatial entropy of the rotor histogram at the first time interval t₁,
θ_{1,t}_{₂}=NPS₂, is the number of phase singularities at the second time interval t₂,
θ_{2,t}_{₂}=RD₂, is the mean rotor duration at the second time interval t₂,
θ_{3,t}_{₂}=SE₂, is the spatial entropy of the rotor histogram at the second time interval t₂, and wherein the reproducibility score (RS) fulfills the following expression:

$$R S = {α}_{1} Δ N P S + {α}_{2} Δ R D + {α}_{3} Δ S E ,$$

wherein a₁, a₂ and a₃ are weighting coefficients and wherein ΔSP, ΔRD and ΔSE are the temporal variabilities of the number of phase singularities, the mean rotor duration and the spatial entropy of the rotor histogram correspondingly.

Such an application is advantageous when considering the reproducibility score (RS) of irregular cardiac arrhythmia, more particularly of atrial fibrillation.

Since the properties of cardiac electrical activity are not stationary during atrial fibrillation, the reproducibility score (RS) is calculated from the mentioned parameters, i.e., NPS, RD and SE, and thus is based on the temporal variability of said metrics, which summarize cardiac electrical activity during the arrhythmia.

In this particular embodiment, the number of phase singularities per time interval NPS, the mean rotor duration RD and the spatial entropy of the rotor histogram SE are calculated at two different time intervals, particularly at the first t₁ and second t₂ time intervals, and their temporal variability (ΔNPS, ΔRD and ΔSE, respectively) are calculated as:

$${Δθ}_{m} = 2 \cdot \frac{\vert {θ}_{m , {t}_{1}} - {θ}_{m , {t}_{2}} \vert}{\vert {θ}_{m , {t}_{1}} + {θ}_{m , {t}_{2}} \vert} ,$$

where θₘ is equal to NPS, RD or SE for computing ΔNPS, ΔRD and ΔSE, respectively.

In the present embodiment, from the estimated epicardial potentials, U_{H}, reentrant activity at the different points of the cardiac surface in a first t₁ and second t₂ time intervals can be summarized using different metrics.

Throughout the present document, reentrant activity shall be understood as cardiac electrical activity which propagates through the cardiac tissue by surrounding an anatomic or functional obstacle, or any other type of obstacle, so that the total propagation time around such obstacle is larger than the refractory period of the cardiac cells (minimum time during which a depolarized cardiac cell cannot be depolarized again), thus resulting in uninterrupted electric propagation.

In a particular embodiment, in steps b) and c), the number of phase singularity (NPS₁, NPS₂) is computed as:

$$N P {S}_{i} = \frac{{\sum }_{t ∈ {t}_{i}} S P ( t )}{{t}_{i}} ,$$

wherein i ∈ [1, 2] and SP(t) are the phase singularity points present along the time t.

A certain node of the cardiac surface is said to be a phase singularity when the epicardial potentials surrounding such node, that is, the epicardial potentials of the adjacent nodes, have a phase progression from −π to π.

In a particular embodiment, the phase singularity points (SP₁,SP₂) are detected over a 2D phase distribution obtained at the first time interval t₁ and at the second time interval t₂ correspondingly by applying a phase transformation to the cardiac electric activity at each node n of the mapped 2D surface.

Thus, singularity points (SP) be detected over 2D phase distributions by employing e.g., methodologies for detecting a phase variation from −π to π around a determined node, such as the detection of the cardiac nodes for which:

$${\sum }_{n ∈ {N}_{neigh}} ∇ {φ}_{n} ( t ) = \pm 2 π ,$$

being N_{neigh} the neighbor nodes of the analyzed node, and ∇φₙ(t) the spatial phase variation from node n to the node n+1 in N_{neigh}. Throughout the present document, the ensemble of neighbor nodes N_{neigh} can be formed by neighbor nodes ranging from 1st to 5th order neighbors of the node of interest.

According to this, the instantaneous phase values distribution in each time interval, first time interval t₁ and at the second time interval t₂, are projected over respective 2D surfaces, so that a 2D phase distribution is obtained for each time interval.

In a particular embodiment, the applied phase transformation is the Hilbert transform, from which phase transformation the phase of U_{H} is computed.

In a particular embodiment, a 2D binary image is obtained from each 2D phase distribution obtained at the first time interval t₁ and at the second time interval t₂, and determining in the 2D binary image whether a concrete pixel is a phase singularity point (SP₁,SP₂).

Advantageously, a phase singularity point (SP₁,SP₂) corresponds to a unique pixel, and not to a plurality of aggregated pixels.

In the particular embodiment of atrial fibrillation, an atrial fibrillation driver is considered to be either a focal or localized source demonstrating fast, repetitive activity that propagates outward from this source, breaking down in to disorganization further away from its origin.

To that end, rotors can be defined as a type of atrial fibrillation drivers, configuring rotational activation patterns which perpetuate asynchronous electrical propagation in the heart.

In a particular embodiment, in steps b) and c), the mean rotor duration (RD₁, RD₂) at the first time interval t₁ and at the second time interval t₂ are obtained by the following steps:
i. stacking 2D binary images in a 3D binary volume,
ii. applying 3D dilation to the 3D binary volume of step i. and connecting the singularity points (SP₁,SP₂) which are close in space or time generating spatiotemporal rotor trajectories,
iii. skeletonizing the spatiotemporal rotor trajectories generated in the 3D dilated binary volume of step ii. to allow the identification of crossing points between different spatiotemporal rotor trajectories,
iv. clustering the different spatiotemporal rotor trajectories, and
v. identifying each cluster as a rotor.

Throughout the present document, singularity points (SP₁,SP₂) which are close in space shall be considered as nodes which can range from 1st to 5th order neighbors of the node of interest, and singularity points (SP₁,SP₂) which are close in time shall be considered as a temporal difference which can range from 5 to 50 ms.

In a particular embodiment, the mean rotor duration RD is obtained as the mean of the time span occupied by the different rotor trajectories.

The transformation of the 3D phase distribution into a 2D phase distribution has the advantage of facilitating the application of filters or any other transformation, which would not be feasible or efficient if working directly on a 3D geometry. Nevertheless any other approach for the detection of singularity points (SP₁,SP₂) or rotors can be applied.

In a particular embodiment, the mean rotor duration RD is one of the M metrics to be considered, in combination with any other metric.

In a particular embodiment, in steps b) and c), a rotor histogram is calculated by counting the number of times that at least one node of the plurality n of nodes of the cardiac geometry surface is a rotor during the first time interval t₁ and the second time interval t₂.

In a particular embodiment, in steps b) and c), the entropy of the rotor histogram (SE₁,SE₂) at the first time interval t₁ and at the second time interval t₂ is obtained by the following expression:

$$S {E}_{i} = - {\sum }_{n = 1}^{N} {p}_{n} {log}_{2} {p}_{n} ,$$

wherein pₙ is the probability of the node n of the plurality of nodes of the cardiac surface to take a given rotor count value, N is the total number of nodes within the cardiac model, and i ∈ [1, 2] is the time interval.

Thus, when the number of rotors is known for each node (N nodes) of the cardiac surface, the probability of the node n of the plurality of nodes of the cardiac surface to take a given rotor count value is the value of the rotor numbers in said particular node, divided by the number of rotors for every node (n nodes).

In a particular embodiment, the weighting coefficients are:

$${α}_{1} = {α}_{2} = {α}_{3} = \frac{1}{3}$$

Thus, the weighting coefficients for the method applied to M=3, that is, to three metrics, is uniform in weight for each of the considered metrics. Thus, the three weights a₁, a₂ and a₃ are equivalent and equal to ⅓, so that ΔSP, ΔRD and ΔSE contribute equally to the calculation of the reproducibility score (RS).

In a particular embodiment, when considering atrial fibrillation, such a repartition of weighting coefficients is advantageous.

In another embodiment, a₁, a₂ and a₃ can have different values, and be computed based on patient characteristics, such as physical characteristics (e.g., sex, body mass index, heart size) or patient condition (e.g., type of arrythmia).

All the features described in this specification (including the claims, description and drawings) and/or all the steps of the described method can be combined in any combination, with the exception of combinations of such mutually exclusive features and/or steps.

### DESCRIPTION OF THE DRAWINGS

These and other characteristics and advantages of the invention will become clearly understood in view of the detailed description of the invention which becomes apparent from a preferred embodiment of the invention, given just as an example and not being limited thereto, with reference to the drawings.

FIG. 1A-1B These figures show, respectively, a block diagram of the acquisition of input data and of the performance of a particular embodiment of the present method.

FIG. 2A-2B These figures show, respectively, a particular embodiment of a cardiac geometry within a torso geometry with a particular surface electrode distribution, and a detailed model of a cardiac geometry corresponding to a detailed view of FIG. 2A.

FIG. 3 This figure shows the estimated electrical activity at each node of the cardiac surface of FIGS. 2A and 2B, corresponding to a first (on the left) and second (on the right) analysis time interval, the first time interval t₁ and at the second time interval t₂, for computation.

FIG. 4A-4B These figures show a graphical representation of the steps for obtaining the singularity points and rotor detection in a particular embodiment of the present method, and an example of a rotor histogram displayed on a 3D bi-atrial model.

FIG. 5 This figure shows the results of a particular embodiment of an atrial segmentation, and the results of the validation of the rotor detection methodology in each of the segmented regions.

FIG. 6 This figure shows the rotor metrics obtained in the particular embodiment of two groups of AF patients, one formed by patients who recovered from AF following PVI and another one formed by patients who experience AF recurrence following PVI. The results are displayed for two different turn threshold for the rotor detection algorithm (0 and 1).

FIG. 7 This figure shows the phase maps and rotor histograms obtained in a particular embodiment at the first time interval t₁ and at the second time interval t₂, for a patient in sinus rhythm (left) and a patient with arrhythmia recurrence (right) following PVI.

FIG. 8 This figure shows mean values between first and second measurements for each of the proposed metrics, i.e. measurements at the first time interval t₁ and at the second time interval t₂, for the patients of FIG. 7.

FIG. 9 This figure shows the mean values of the absolute difference between first and second measurements for each of the proposed metrics, i.e. measurements at the first time interval t₁ and at the second time interval t₂, for the patients of FIG. 7.

### DETAILED DESCRIPTION OF THE INVENTION

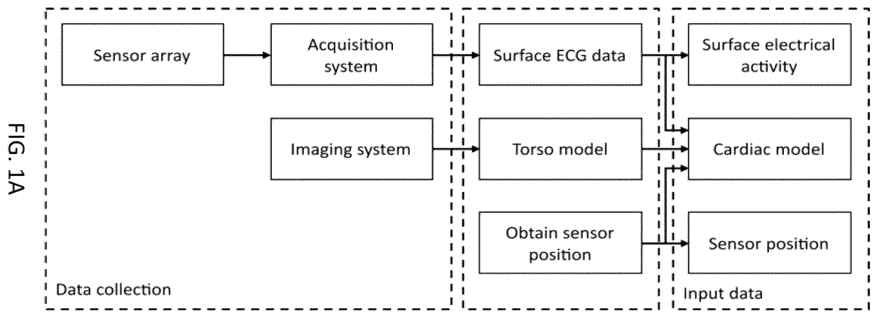

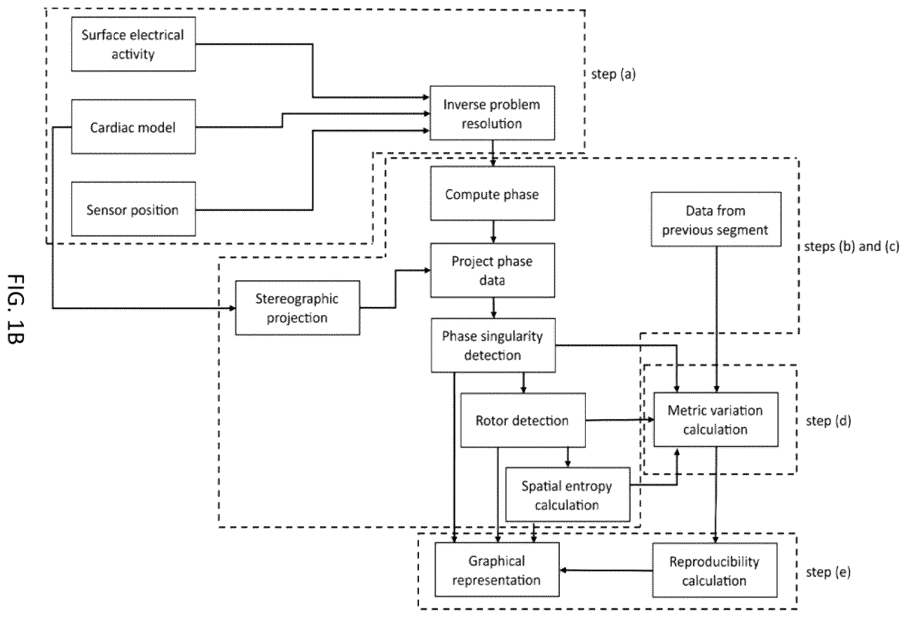

FIG. 1A shows a block diagram of the acquisition of the input data required in a particular embodiment of step a) of the present method.

As shown, data collection is performed in a particular embodiment by means of a sensor array, i.e. a distribution of electrodes laid up according to a particular distribution. Additionally, an acquisition system is required for receiving the data from the mentioned sensor array. Moreover, an imaging system is also used in the data collection, particularly an imaging system which allows taking images of the torso of the patient and/or of the surface of the heart of the patient.

As shown in FIG. 1A, the sensor array and acquisition system provide surface ECG data, whilst the imaging system allows obtaining a torso model of the patient.

The combination of the mentioned data is considered the input data required in a particular embodiment of step a) of the present method, data with which a cardiac model and the electrical activity at the cardiac surface is obtained.

Particularly, the sensor distribution is an array which is placed on the torso of a patient suffering from an arrhythmia, particularly atrial fibrillation. In a particular embodiment, such sensor array comprises 128 surface ECG electrodes homogeneously distributed over the patient's torso.

The sensor array is connected to an acquisition system, particularly a biosignals amplifier which acquires and digitizes the surface ECG signals captured by the sensor array. In addition, a 3D model of the patient's torso is obtained by using a 3D imaging system allowing to capture images of the patient's torso from different perspectives.

The position of each sensor of the sensor array is obtained by applying image recognition techniques to the images captured by the 3D imaging system.

The surface ECG data, the 3D torso model and the sensor position are employed to estimate the most appropriate surface cardiac geometric model for the patient.

In a particular embodiment, the cardiac geometric model corresponds to an atrial model, and ventricular activity suppression (i.e., QRST suppression) techniques are applied to the surface ECG signals in order to preserve only atrial electrical activity.

FIG. 1B shows a particular embodiment of the performance of the method of the invention, wherein input data obtained in FIG. 1A is used in step a) of said method.

Particularly, the information present in the cardiac model obtained is used for an inverse problem resolution in step a) of the method.

Afterwards, two different time intervals, the first time interval t₁ and the second time interval t₂, are selected within the atrial electrical signals for its further analysis.

Steps b) and c) of the method provide the computing and projection of the phase data obtained from the inverse problem resolution.

Particularly, the cardiac model used in step a) is transformed into a 2D flat surface by applying a conformal map projection, particularly a stereographic projection, and the ECGI signals at the first time interval t₁ and at the second time interval t₂, are obtained by solving the inverse problem, using also as inputs the atrial electrical activity at the first time interval t₁ and at the second time interval t₂, the torso and cardiac models and the sensors position already acquired.

Phase of the ECGI signals at the first time interval t₁ and at the second time interval t₂, and at each node of the cardiac model are obtained by applying a phase transformation, particularly a Hilbert's transform, to said signals.

For each time interval, t₁ and t₂, a phase matrix is obtained, with the values in each row corresponding to the phase at a different node of the cardiac model and the values in each column corresponding to the temporal evolution of the phase at each node.

These phase matrixes are mapped to the 2D flat representation of the cardiac model to obtain 2D phase distributions. In these 2D phase distributions, the singularity points SPs are detected, and the number of singularity points NSPs is computed for said time intervals t₁ and t₂.

Then, a rotor detection is performed, which is used for the computation of the spatial entropy of the rotor histogram.

Thus, the computed metrics of the present embodiment are the number of singularity points NSP, the rotors RD and the spatial entropy of the rotor histogram SE.

Step d) of the present embodiment provides the metric variation calculation, i.e. the temporal variability of NSP, RD and SE (ΔSP, ΔRD and ΔSE, respectively). Such metric variation is computed for the time intervals t₁ and t₂.

These values provide information on how repetitive the quantified metrics are, and allow to establish confidence intervals by thresholding the allowed temporal variation.

From the individual temporal variation of each of the considered metrics, a reproducibility score, RS, is obtained in step e), particularly in the present embodiment according to the following expression:

$$R S = \frac{1}{3} Δ S P + \frac{1}{3} Δ R D + \frac{1}{3} Δ S E$$

Finally, step e) as shown in present FIG. 1B provides graphical representation of the aforementioned computed metrics, combined with the calculated reproducibility score RS.

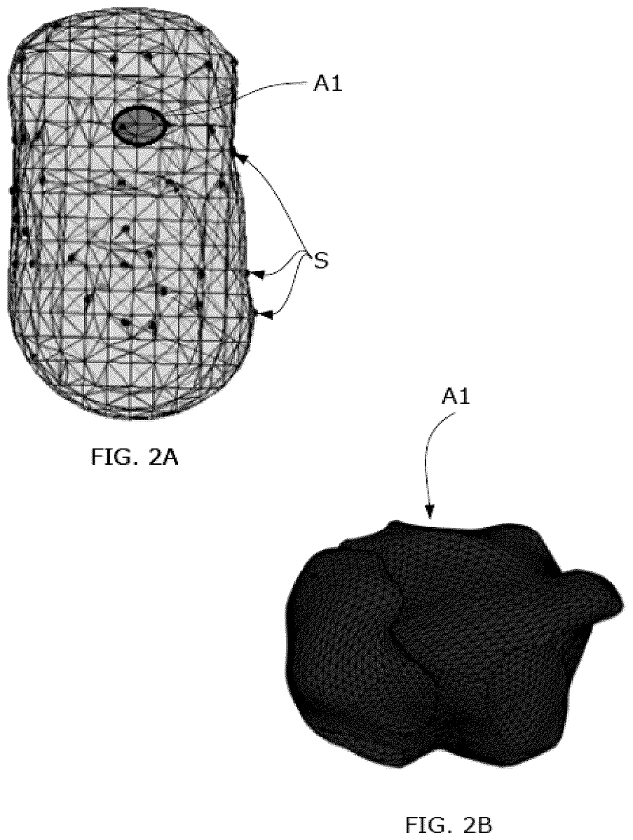

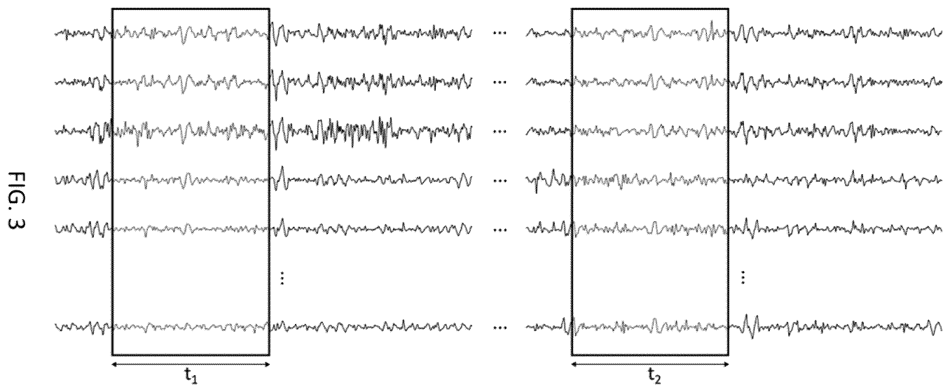

FIG. 2A shows a particular embodiment of a cardiac geometry (A1) within a torso geometry. Such cardiac geometry (A1) is highlighted in the torso geometry acquired.

Furthermore, the torso geometry comprises several sensors (S) or electrodes, which are distributed in a predetermined manner according to the acquisition of electrical activity which is performed on the mentioned torso.

FIG. 2B shows the detailed model of the cardiac geometry (A1) highlighted in FIG. 2A.

FIG. 3 shows two different graphics, corresponding to the graphics at the first time interval t₁, shown at the left side of the figure, and at the second time interval t₂, shown at the right side of the figure, of the estimated electrical activity at each of the selected nodes.

Particularly, each of the rows of each of the graphics correspond to the electrical activity of one particular node of the ones selected for the acquisition of the electrical activity.

Both the first time interval t₁ and at the second time interval t₂, have been highlighted at the graphics by means of a rectangle which shows the duration of each time interval and the electrical activity corresponding to each of the nodes during such time interval.

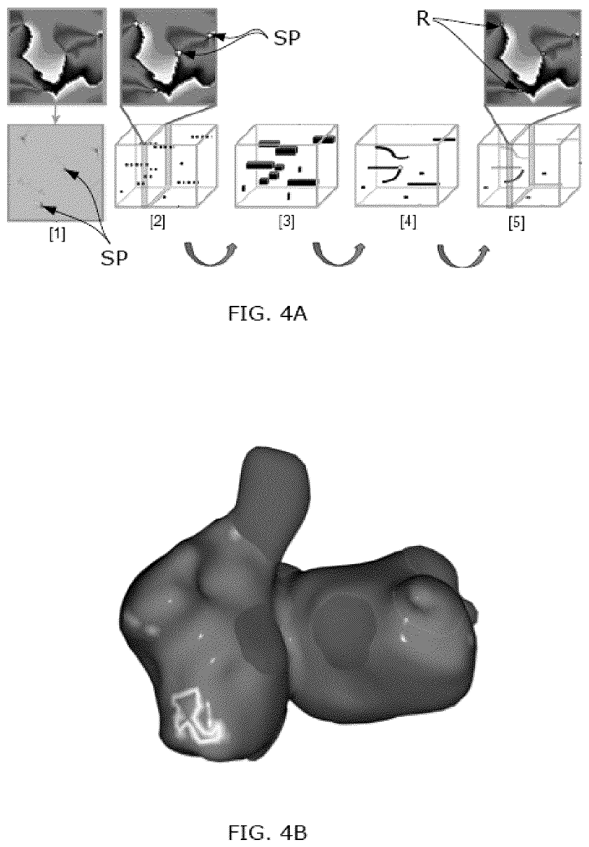

FIG. 4A shows the steps performed for obtaining the singularity points and rotor detection during steps b) and c) of the present embodiment of the method.

Particularly, at first, in section [1], a topological charge method is performed, by departing from a volume generated by stacking instantaneous 2D phase maps which are obtained from each analysis segment.

That is, the upper image of section [1] corresponds to a 2D phase distribution whereas the lower image of section [1] corresponds to the 2D binary image, wherein the singularity points (SP) are detected, excluding those singularity points (SP) which do not present a linear phase progression in their surroundings.

The detected singularity points (SP), shown in the lower image of section [1], are coded in a volume of stacked binary images, representing the spatiotemporal location of each singularity point (SP), as shown in section [2].

Particularly, section [2] corresponds to the obtention of the phase singularities in a 3D volume. That is, the upper image of section [2] shows the 2D phase distribution with the singularity points (SP), being said upper image a detailed image of the lower image of section [2], which shows the 3D volume and, as black squares highlighted therein, the singularity points (SP). Therefore, a binary volume is shown in said lower image of section [2].

Section [3] shows a 3D binary dilation, or dilated volume, which is applied to the binary volume of section [2], in order to connect neighbor singularity points (SP) and eliminate possible gaps due to misdetections, as shown in the image by means of solid black parallelepipeds.

However, this process might artificially connect unrelated rotor trajectories, thus introducing crossing points.

In order to identify said crossing points, the dilated volume of section [3] is skeletonized as shown in section [4], the existing crossing points being all detected.

According to said skeletonizing, the rotor (R) trajectories converging at a crossing point are split into different trajectories. On the other hand, in the case that the offset and onset of consecutive rotor (R) trajectories was very close in time, both trajectories were merged into a single one, as shown in the image corresponding to section [4].

Finally, section [5] shows a trajectory clustering, wherein the number of turns of each rotor (R) is quantified as the number of times that the phase varies from −π to π along the rotor (R) trajectory.

The upper image of section [5] shows, in a 2D phase distribution, the rotors (R) as a detailed image of the 3D skeletonized volume of section [4].

FIG. 4B shows a rotor histogram displayed on a 3D bi-atrial model according to the rotor detection shown in FIG. 4A.

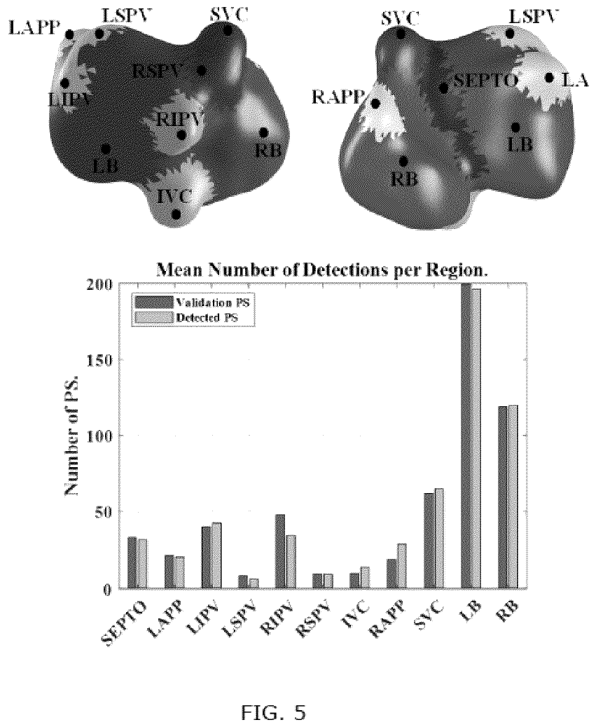

FIG. 5 shows an atrial segmentation, particularly the image of the left side of the figure shows the postero-anterior view of an atrial model, whereas the image on the right side of the figure shows the antero-posterior view of the same atrial model.

Additionally, the graphic on the bottom part of the figure shows the validation of the rotor detection algorithm, showing the mean number of singularity points (SP) per region of each of the recordings used in the performed validation.

As shown, the validation singularity points (SP) are shown as the left column of each region, whereas the detected singularity points (SP) are shown as the right column of each region.

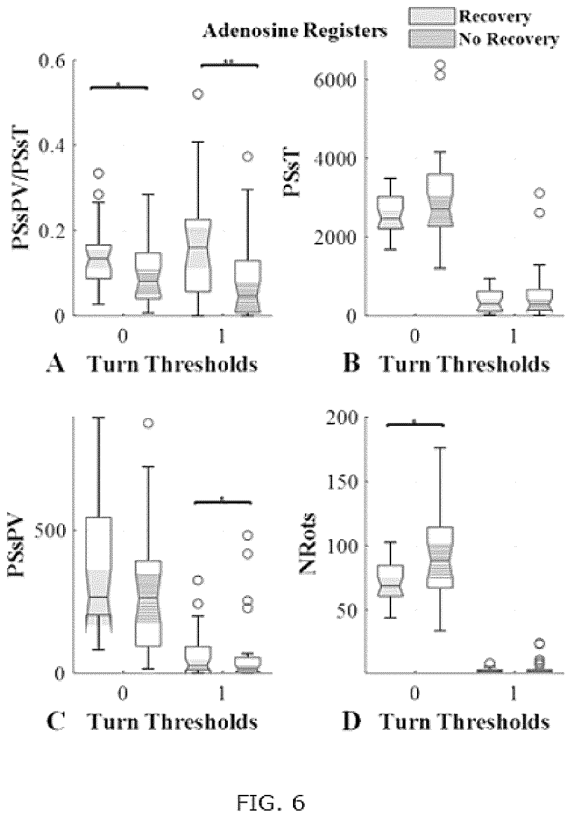

FIG. 6 shows 4 different graphics, containing the rotor metrics computed in the aforementioned example, regarding AF patients, specifically with (*p<0.05; **p<0.01).

Particularly, graphic A shows the ratio of singularity points (SP) found in PPVV over the singularity points (SP) in the whole atria. Graphic B shows the number of singularity points (SP) per second in the atria.

Additionally, graphic C shows the number of singularity points (SP) in the PPVV per second, whereas graphic D shows the number of rotors in the atria per second.

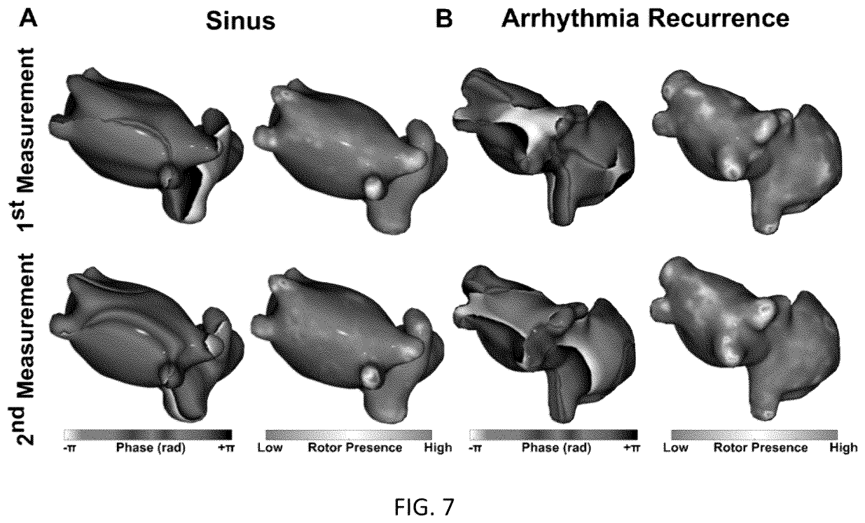

FIG. 7 shows the phase maps and rotor histograms obtained in the same embodiment of an AF patient, particularly the phase maps and rotor histograms obtained at the first time interval t₁ and at the second time interval t₂-Particularly, it is directed to a patient in sinus rhythm 6 months after PVI (A), on the left side of the image, and a patient with arrhythmia recurrence (B) on the right side of the image.

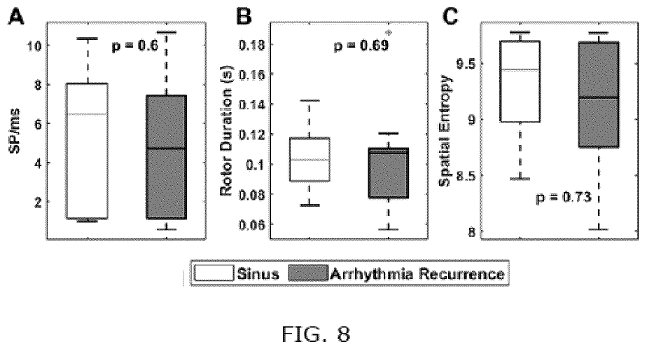

FIG. 8 shows the mean values between first and second measurements for each of the proposed metrics for the patients described in FIG. 7.

That is, the presented graphics show the mean values between the measurements performed at the first time interval t₁ and at the second time interval t₂, wherein the measurements performed correspond to the computed metrics, i.e. number of singularity points (SP), rotors (RD) and spatial entropy of the rotor histogram (SE).

In FIG. 8, results in white correspond to a patient in sinus rhythm, whereas results in gray correspond to a patient with arrhythmia recurrence.

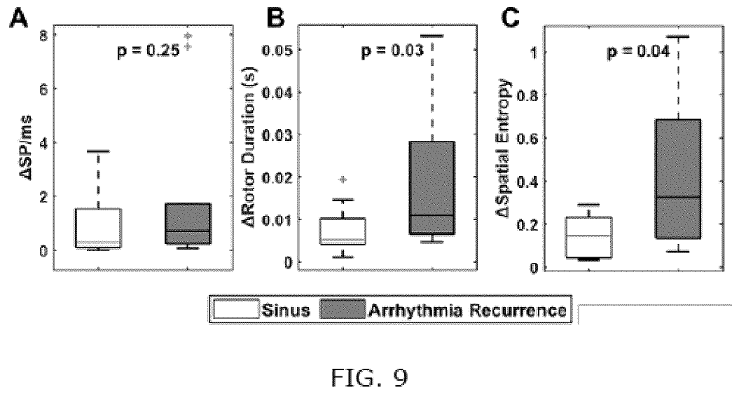

FIG. 9, as FIG. 8, shows results in white corresponding to a patient in sinus rhythm, whereas results in gray correspond to a patient with arrhythmia recurrence.

In particular, the present figure shows the mean values of the absolute difference between first and second measurements for each of the proposed metrics, i.e. measurements at the first time interval t₁ and at the second time interval t₂-Again, the measurements performed correspond to the computed metrics, i.e. number of singularity points (SP), rotors (RD) and spatial entropy of the rotor histogram (SE).

For a better description of the present invention, particular examples are also disclosed below as embodiments of the present method, wherein in example 1, corresponding to an AF arrhythmia, three metrics have been computed, as in the embodiment disclosed by means of the aforementioned figures.

Example 2 corresponds to a particular embodiment wherein, although being related also to an AF arrhythmia, three metrics have been computed being only two of them finally relevant for the calculation of the reproducibility score (RS) obtained when the proceeding is performed.

### Example 1: Rotor Detection and Evaluation on the Clinical Outcome Prediction of Rotor Detection in Non-Invasive Phase Maps in Patients with Atrial Fibrillation (AF)

The aim of the present example was to apply the rotor detection method of the present invention in order to evaluate its performance and determine the capability of different rotor metrics to predict the clinical outcome of atrial fibrillation (AF) patients following pulmonary vein isolation (PVI).

Regarding the data acquisition as shown in FIG. 1A, the torso surface ECG signals is acquired at 57 locations by means of 57 electrodes, in a total number of 29 AF patients scheduled for PVI, following adenosine infusion administration.

Torso geometry of each patient was reconstructed by applying photogrammetry to a video recording of each of the patient's torso, and the electrode positions were manually annotated by the operator. Atrial geometries were obtained from MRI/CT scan images obtained prior to the intervention.

By means of the mentioned techniques, input data required for step a) of the present method was obtained.

Patients were followed-up 6 months after the intervention, and they were grouped attending to the successfulness of the intervention to maintain sinus rhythm. 15 patients were at sinus rhythm 6 months after the intervention, whereas 14 had experienced arrhythmia recurrence.

In particular, for the performance of the method of the present invention as shown in FIG. 1B, two signal segments per patient were selected (4.06±0.311 s) for its analysis (with the exception of 1 subject, for who only one segment was available), that is, the first time interval t₁ and at the second time interval t₂ are selected as identical time intervals.

In step a) of the method, ECGI signals were obtained by solving the inverse problem using zero-order Tikhonov regularization and L-curve optimization for each segment.

Then, steps b) and c) were performed by following the obtention of the 3D ECGI voltage maps on the atrial geometry, which were converted to 2D squared images using conformal mapping, and the instantaneous phase of the 2D voltage distribution was obtained by computing the Hilbert's Transform.

In addition, a validation set for the proposed rotor detection algorithm is generated also in steps b) and c). For this purpose, 9 segments in which reentrant activity can be visually identified were selected among all the analyzed segments. The location of the phase singularities (SP) was defined as pixels in the 2D image for which the surrounding pixels present a phase progression from −π to π, and is particularly performed manually by a trained researcher.

That is, singularity points (SP) were defined as those points of the atrial geometry around which there is a stable electrical activity reentry (phase progressions from −π to π) during at least one complete rotation, and in at least two of three concentric rings, as described before.

As shown in FIG. 4A, a stack of the instantaneous 2D phase maps obtained for each analysis segment was performed, and the singularity points (SP) are detected in such volume by means of the topological charge method, excluding those singularity points (SP) which did not present a linear phase progression in their surroundings.

The detected singularity points (SP) were coded in a volume of stacked binary images, representing the spatiotemporal location of each singularity point (SP).

Afterwards, a 3D binary dilation was applied to the binary volume, in order to connect neighbor singularity points (SP) and eliminate possible gaps due to misdetections.

The dilated volume was skeletonized and all the existing crossing points were detected, thus obtaining rotor (R) trajectories which converge at a crossing point that were split into different trajectories. In the case that the offset and onset of consecutive rotor (R) trajectories is very close in time, both trajectories were merged into a single one.

Rotors were defined as singularity points (SP) which can be connected in time (i.e, which maintain a spatio-temporal causality), and rotor histograms were generated by counting the number of times that each node of the atrial model was considered to be a rotor (R).

Shannon spatial entropy (SE) was computed on the rotor histogram.

Finally, the number of turns of each rotor was quantified as the number of times that the phase varied from −π to π along the rotor (R) trajectory.

The performance of the proposed rotor detection algorithm is assessed by computing its precision (P), recall (R), and F_{β} score in the detection of the manually labeled singularity points (SP). The F_{β} score is a metric summarizing both P and R as follows:

$${F}_{β} = ( 1 + {β}^{2} ) \cdot \frac{P \cdot R}{{β}^{2} \cdot P + R}$$

In the present example, β was set to 2.

Additionally, several rotor-related metrics were computed, namely the number of rotors per second, the number of singularity points (SP) in the pulmonary veins (PPVV) per second, the number of singularity points (SP) in the whole atria per second, and the ratio between the total number of singularity points (SP) detected in the PPVV and in rest of the atria. All these values were computed for all the detected rotors (0 turn threshold), and also excluding those rotors with less than 1 turn (1 turn threshold).

The median metrics obtained for the two outcome groups were compared by using the Mann-Whitney U test, setting the significance level at p-value <0.05.

Regarding the rotor detection algorithm validation, Table 1 shown below summarizes the recall, precision and F-score obtained in all the recordings of the validation set (mean values of 0.75, 0.82 and 0.75, respectively).

TABLE 1
Recall, Precision and F-score values obtained in the validation data set.

| Recall | Precision | F-score |
|---|---|---|
| 0.9191 | 0.74 | 0.8787 |
| 0.74 | 0.70 | 0.73 |
| 0.71 | 1.00 | 0.76 |
| 0.74 | 0.76 | 0.74 |
| 0.81 | 0.63 | 0.76 |
| 0.62 | 0.77 | 0.65 |
| 0.54 | 1.00 | 0.60 |
| 0.82 | 0.80 | 0.81 |
| 0.84 | 0.97 | 0.87 |

The performance in the detection of singularity points (SP) in the different atrial regions was the one shown in FIG. 5. The regions presenting largest and lowest relative errors are the right inferior and right superior PPVV (RIPV and RSPV), respectively. It can be also noticed that the left and right atrial bodies (LB and RB) present a much higher number of both labelled and detected singularity points (SP), due to the fact that these regions are also larger than the rest. With respect to the PPVV, a larger number of rotors are detected in the inferior (left/right inferior PPVV, LIPV/RIPV) than in the superior (left/right superior PPVV, LSPV/RSPV) veins.

Additionally, FIG. 6 shows the results for patients for which PVI was successful and presented a higher number of singularity points (SP) in the PPVV than those with recurrent arrhythmia (median of 26.28 vs 12.16, p<0.05), and also a higher ratio of singularity points (SP) in the PPVV with respect to the rest of the atrial surface (median of 0.16 vs 0.04, p<0.01). On the contrary, no differences between groups were observed for the total number singularity points (SP).

Regarding the number of rotors, it was shown to be lower for patients at sinus rhythm (group A) than for those with arrhythmia recurrence (group B) when no turn threshold was applied (88.21 vs 68.55, p<0.05) but no differences were observed when a threshold of 1 turn was applied.

Thus, the present example shows results which suggest a high performance of the method of the invention for rotor detection, and emphasizes the fact that rotor-related metrics obtained from non-invasive ECGI signal analysis are useful for the prediction of the clinical outcome of PVI.

Therefore, the analysis of these metrics could contribute to a better selection of the most appropriate treatment strategy for each patient.

### Example 2: Reproducibility of Phase Derived Metrics from EGCI in Atrial Fibrillation

The aim of the present example was to apply the methodology disclosed in the present invention for the calculation of a reproducibility score in order to determine whether the reproducibility of phase derived metrics obtained from ECGI signals is related with the complexity of the arrhythmia in atrial fibrillation (AF) patients, and also to evaluate the relationship of said metrics with the outcome of pulmonary vein isolation (PVI).

First of all, regarding the necessary data collection, in the present example the torso surface ECG signals were acquired at 57 locations (with 57 electrodes) in 24 AF patients (61.8±14.3 years; 6 males and 18 females; 13 paroxysmal AF and 11 persistent AF) scheduled for PVI and valvuloplasty.

According to the mentioned data, the torso geometry of each patient was reconstructed by applying photogrammetry to a video recording of the patient's torso, and electrode position was manually annotated by the operator. Atrial geometries were obtained from MRI/CT scan images obtained prior to the intervention.

Patients were followed-up 6 months after the intervention, and they were grouped attending to the successfulness of the intervention to maintain sinus rhythm, as in the example before. Particularly, 13 patients were at sinus rhythm 6 months after the intervention (group A), whereas 11 had experienced arrhythmia recurrence (group B).

With the input data, signal segments corresponding to two different time intervals were analyzed for each patient during AF. Raw surface ECG signals corresponding to said time intervals were band-pass filtered with a 10^{th} order Butterworth filter and cut-off frequencies of 2 and 45 Hz, after which ventricular activity was removed by computing the principal component analysis of the average beat of the segment. Additionally, those components reflecting ventricular activity from each beat were subtracted.

ECGI signals were obtained by solving the inverse problem using zero-order Tikhonov regularization and L-curve optimization for each segment, as in the previous example. Then, instantaneous phase was also obtained by computing the Hilbert's Transform of each signal.

As before, singularity points (SP) were defined as those points of the atrial geometry around which there is a stable electrical activity reentry (phase progressions from −π to π) during at least one complete rotation, and in at least two of three concentric rings, as described before, and rotors (R) are defined as singularity points (SP) which can be connected in time (i.e., which maintain a spatio-temporal causality), and rotor histograms were generated by counting the number of times that each node of the atrial model is considered to be a rotor. Shannon spatial entropy (SE) was computed on the rotor histogram.

As indicated, three different metrics were obtained for each of the two analyzed segments of each patient, namely:
The total number of singularity points (SP) in each interval, normalized by the interval duration (SP/ms);
The mean duration of the detected rotors (RD); and
The Shannon spatial entropy (SE) of the rotor histogram.

In order to evaluate the reproducibility of each of the previous metrics, the absolute differences between each metric obtained at each of the two analyzed time intervals, namely the first time interval t₁ and the second time interval t₂, were obtained (ΔSP/ms, ΔRD and ΔSE for the differences in SP/ms, RD and SE, respectively).

Additionally, the coefficient of determination (R²) between the first and second metrics, that is, between the metrics at the first time interval t₁ and at the second time interval t₂, was also computed.

As mentioned before, a statistical analysis was performed, thus obtaining mean values of each metric which was computed at each time interval (t₁ and t₂), and normality in data distribution in each patient group was determined using a Kolmogorov-Smirnov test.

In order to evaluate differences between patients at sinus rhythm (group A) or presenting arrhythmia recurrence (group B), a Wilcoxon rank-sum test was applied to compare non-normally distributed samples, whilst Student's t-test was applied to normally distributed samples. Statistical differences in the R² between groups were computed using a tail t-test after Fisher r-to-z transform.

In all the cases, significance level was set at p-value <0.05.

The outcome prediction based on ECGI reproducibility was obtained, by means of the reproducibility score (RS) which was computed from the differences of some of the previously defined metrics among intervals as follows:

$$R S = \frac{1}{2} Δ R D + \frac{1}{2} Δ S E$$

As shown, the metric of the number of singularity points (SP) was finally not considered in the calculation of the reproducibility score (RS).

That is, univariate logistic regression was applied to RS in order to determine whether this index can be used to discriminate PVI success. The same regressor was also computed using the AF type (paroxysmal vs persistent) as a predictor, to compare the performance of the RS with that of the current clinical practice, which is based on the scheduling of the PVI according to the type of AF. Performance of both approaches was compared by computing the area under the curve (AUC), sensitivity and specificity of each of them.

### Temporal Reproducibility of ECGI Derived Metrics

The phase maps and rotor histograms obtained for two sample patients, one at sinus rhythm and the other with arrhythmia recurrence 6 months following the PVI procedure, were shown in FIG. 7-9 as disclosed. In both cases, reentries can be observed, with a higher reentry prevalence in the pulmonary veins. It can be noticed how the phase map of the patient with a bad PVI outcome have a more complex pattern, thus reflecting a more heterogeneous propagation than that observed in the maps of the patient who underwent a successful intervention. It can be also observed how the rotor histograms are more repeatable for the patient for who PVI was effective.

Particularly in FIG. 8, the values distribution of the different proposed metrics obtained at one analysis interval are displayed for each of the groups of interest. None of the metrics were able to anticipate the outcome of PVI. Nevertheless, the analysis of a given metrics at the two different analysis intervals revealed higher R² values in all of them for the group of patients with a successful PVI than for those who experienced arrhythmia recurrency. In this way, the values of R2 were of 0.87 vs. 0.36 (p=0.04) for SP/ms, 0.82 vs. 0.62 (p>0.05) for RD, and 0.87 vs. 0.39 (p=0.05) for SE.

Moreover, ΔSP/ms, ΔRD and ΔSE presented a similar tendency than the R² values, ΔRD and ΔSE showing significant differences among groups, as shown in FIG. 9.

PREDICTION of PVI success based on ECGI variability metrics

In the classification of PVI outcome using logistic regression, AUC of 0.77 was obtained when RS was employed as the predicting variable, whereas this value decreased to 0.59 when the arrhythmia type is employed.

This result was supported when analyzing the sensitivity (0.64 vs 0.63) and specificity (0.85 vs 0.64) of both methods.

Therefore, the use of RS outperforms the current clinical practice, and the study of arrhythmia reproducibility represents a potential tool for a personalized and better selection of the therapeutic approach for each patient.

That is, reproducibility of ECGI derived metrics during AF is higher for patients who are at sinus rhythm 6 months after PVI (group A) than for those with arrhythmia recurrence (group B). The analysis of reproducibility according to the present invention is thus of great help in the selection of the most adequate therapeutic approach for each patient.

1. A computer implemented method for obtaining the reproducibility score (RS) of cardiac arrhythmia, the method comprising the following steps:
a) collecting as input data a geometrical model of a cardiac surface of a patient, the cardiac surface comprising a plurality N ∈ [symbol] of nodes, and the cardiac electrical activity at the plurality N of nodes of said cardiac surface of a patient,
b) computing from the input data of step a), during a first time interval t₁, a number M of metrics designated as θ_{m,t}_{₁}, wherein m=1, . . . M,
c) computing from the input data of step a), during a second time interval t₂ which does not overlap with the first time interval t₁, the M metrics designated as θ_{m,t}_{₂}, wherein m=1, . . . M,
d) determining the temporal variability Δθₘ of each of the M metrics computed, whereinm=1, . . . M, and
e) obtaining a reproducibility score (RS) as a combination of the temporal variability Δθₘ of each of the M metrics computed, wherein m=1, . . . M, being each temporal variability Δθₘ weighted by a corresponding weighting coefficient.

2. The computer implemented method for obtaining the reproducibility score (RS) of cardiac arrhythmia according to claim 1, wherein the weighting coefficients for each of the M computed metrics are calculated based on patient characteristics, such as physical characteristics or patient condition.

3. The computer implemented method for obtaining the reproducibility score (RS) of cardiac arrhythmia according to claim 1, wherein the number of metrics M employed for computing the reproducibility score (RS) is greater than 1.

4. The computer implemented method for obtaining the reproducibility score (RS) of cardiac arrhythmia according to claim 1, wherein in step e) the reproducibility score (RS) is obtained by a n^{th} order polynomic combination of the temporal variability Δθₘ of each of the M metric computed, weighted by respective weighting coefficients.

5. The computer implemented method for obtaining the reproducibility score (RS) of cardiac arrhythmia according to claim 1, wherein in step e) the reproducibility score (RS) is obtained by combining the temporal variability Δθₘ of each of the M metric computed using neural networks.

6. The computer implemented method for obtaining the reproducibility score (RS) of cardiac arrhythmia according to claim 1, wherein the reproducibility score (RS) of step e) is obtained according to the following expression:
$$R S = {α}_{1} Δ {θ}_{1} + {α}_{2} Δ {θ}_{2} + ⋯ + {α}_{M} Δ {θ}_{M} ,$$
wherein aₘ is the weighting coefficient for the m^{th} metric and Δθₘ is the temporal variability of the m^{th} metric computed.

7. The computer implemented method for obtaining the reproducibility score (RS) of cardiac arrhythmia according to claim 1, wherein at least one temporal variability Δθₘ of the m^{th} metric is obtained by one of the following expressions:
$$Δ {θ}_{m} = \vert {θ}_{m , {t}_{1}} - {θ}_{m , {t}_{2}} \vert , or$$
$${Δθ}_{m} = \frac{\vert {θ}_{m , {t}_{1}} - {θ}_{m , {t}_{2}} \vert}{\vert {θ}_{m , {t}_{1}} \vert} , or$$
$${Δθ}_{m} = \frac{\vert {θ}_{m , {t}_{1}} - {θ}_{m , {t}_{2}} \vert}{\vert {θ}_{m , {t}_{2}} \vert} , or$$
$${Δθ}_{m} = {\vert {θ}_{m , {t}_{1}} - {θ}_{m , {t}_{2}} \vert}^{p} , p ∈ , or$$
$${Δθ}_{m} = 2 \cdot \frac{\vert {θ}_{m , {t}_{1}} - {θ}_{m , {t}_{2}} \vert}{\vert {θ}_{m , {t}_{1}} + {θ}_{m , {t}_{2}} \vert} ,$$
wherein θ_{m,t}_{₁} and θ_{m,t}_{₂} are the values of the m^{th} metric obtained from the first and second time intervals, t₁ and t₂, respectively.

8. The computer implemented method for obtaining the reproducibility score (RS) of cardiac arrhythmia, according to claim 1, wherein M=3, and wherein:
θ_{1,t}_{₁}=NPS₁, is a number of phase singularities at the first time interval t₁,
θ_{2,t}_{₁}=RD₁, is a mean rotor duration at the first time interval t₁,
θ_{3,t}_{₁}=SE₁, is a spatial entropy of the rotor histogram at the first time interval t₁,
θ_{1,t}_{₂}=NPS₂, is a number of phase singularities at the second time interval t₂,
θ_{2,t}_{₂}=RD₂, is a mean rotor duration at the second time interval t₂,
θ_{3,t}_{₂}=SE₂, is a spatial entropy of the rotor histogram at the second time interval t₂, and wherein the reproducibility score (RS) fulfills the following expression:
$$R S = {α}_{1} Δ S P + {α}_{2} Δ R D + {α}_{3} Δ S E ,$$
wherein a₁, a₂ and a₃ are weighting coefficients and wherein ΔSP, ΔRD and ΔSE are the temporal variabilities of the number of phase singularities, the mean rotor duration and the spatial entropy of the rotor histogram correspondingly.

9. The computer implemented method for obtaining the reproducibility score (RS) of cardiac arrhythmia according to according to claim 1, wherein the cardiac electrical activity at the plurality N of nodes of step a) is obtained by solving an inverse problem on a torso geometry model of a patient.

10. The computer implemented method for obtaining the reproducibility score (RS) of cardiac arrhythmia according to claim 9, wherein the torso geometry model of a patient is obtained by a nonmedical imaging system.

11. The computer implemented method for obtaining the reproducibility score (RS) of cardiac arrhythmia according to claim 1, wherein the cardiac electrical activity at the plurality N of nodes of step a) is collected with at least one catheter placed within the cardiac chambers.

12. The computer implemented method for obtaining the reproducibility score (RS) of cardiac arrhythmia according to claim 1, wherein the geometric model of the cardiac surface of step a) is obtained by segmentation of medical images or by adjustment of a mathematical model.

13. The computer implemented method for obtaining the reproducibility score (RS) of cardiac arrhythmia according to claim 1, wherein the cardiac surface comprising the plurality N of nodes in step a) is mapped to a 2D surface comprising the plurality of nodes by means of conformal mapping.

14. The computer implemented method for obtaining the reproducibility score (RS) of cardiac arrhythmia according to claim 8, wherein in steps b) and c), the number of phase singularity (NPS₁, NPS₂) is computed as:
$$N P {S}_{i} = \frac{{\sum }_{t ∈ {t}_{i}} S P ( t )}{{t}_{i}} ,$$
wherein i ∈ [1, 2] and SP(t) are the phase singularity points present along the time instant t.

15. The computer implemented method for obtaining the reproducibility score (RS) of cardiac arrhythmia according to claim 14, wherein:
the cardiac surface comprising the plurality N of nodes in step a) is mapped to a 2D surface comprising the plurality of nodes by means of conformal mapping; and
the phase singularity points (SP₁, SP₂) are detected over a 2D phase distribution obtained at the first time interval t₁ and at the second time interval t₂ correspondingly by applying a phase transformation to the cardiac electric activity at each node n of the mapped 2D surface.

16. The computer implemented method for obtaining the reproducibility score (RS) of cardiac arrhythmia according to claim 15, wherein a 2D binary image is obtained from each 2D phase distribution obtained at the first time interval t₁ and at the second time interval t₂, and wherein in the 2D binary image it is determined whether a concrete pixel is a phase singularity point (SP₁, SP₂).

17. The computer implemented method for obtaining the reproducibility score (RS) of cardiac arrhythmia claim 16, wherein in steps b) and c), the mean rotor duration (RD₁, RD₂) at the first time interval t₁ and at the second time interval t₂ are obtained by the following steps:
i. stacking 2D binary images in a 3D binary volume,
ii. applying 3D dilation to the 3D binary volume of step i. and connecting the singularity points (SP₁,SP₂) which are close in space or time generating spatiotemporal rotor trajectories,
iii. skeletonizing the spatiotemporal rotor trajectories generated in the 3D dilated binary volume of step ii. to allow the identification of crossing points between different spatiotemporal rotor trajectories,
iv. clustering the different spatiotemporal rotor trajectories, and
v. identifying each cluster as a rotor.

18. The computer implemented method for obtaining the reproducibility score (RS) of cardiac arrhythmia according to claim 8, wherein in steps b) and c), a rotor histogram is calculated by counting the number of times that at least one node of the plurality N of nodes of the cardiac surface is a rotor during the first time interval t₁ and the second time interval t₂.

19. The computer implemented method for obtaining the reproducibility score (RS) of cardiac arrhythmia according to claim 18 wherein in steps b) and c), the spatial entropy of the rotor histogram (SE₁, SE₂) at the first time interval t₁ and at the second time interval t₂ is obtained by the following expression:
$$S {E}_{i} = - {\sum }_{n = 1}^{N} {p}_{n} {log}_{2} {p}_{n} ,$$
wherein pₙ is the probability of the node n of the plurality of nodes of the cardiac surface to take a given rotor count value, N is the total number of nodes within the cardiac model, and i ∈ [1, 2] is the time interval.

20. The computer implemented method for obtaining the reproducibility score (RS) of cardiac arrhythmia according to claim 1, wherein the first and second time intervals, t₁ and t₂, are non-consecutive time intervals.
